(19) United States
(12) Patent Application Publication
(10) Pub. No.: US 2003/0185884 A1
(43) Pub. Date: Oct. 2, 2003

(54) Therapeutic agent delivery compositions for buccal cavity absorption of pilocarpine

(76) Inventors: Nikhilesh Nihala Singh, Irvine, CA (US); Natasha Nikhilesh Singh, Irvine, CA (US)

Correspondence Address: David J. Meyer, Esq., Manatt, Phelps & Phillips, LLP, 11355 West Olympic Boulevard, Los Angeles, CA, 90064, US

(21) Appl. No.: 10/113,088

(22) Filed: Apr. 1, 2002

(51) Int. Cl. A61K 9/68; A61K 31/555; A61K 31/4178

(52) U.S. Cl. 424/465; 514/397; 514/184

(57) ABSTRACT

A therapeutic agent delivery composition containing a functional buffer system for facilitating absorption of pilocarpine in the buccal cavity. More particularly, the composition is used to raise the pH of the buccal cavity so that the pilocarpine agent is delivered in a lipophilic form capable of crossing the buccal membrane. The delivery composition raises the pH of the buccal cavity through the implementation of a buffer system, which comprises a combination of two or more buffer agents. The agents of the buffer system are combined in a manner where the stronger basic agent is at relatively larger levels than that of a corresponding weaker base. The delivery composition may be administered as a chewing gum, where the pilocarpine is effectively absorbed in about 5 minutes after the onset of chewing. In addition, a pharmacologically effective concentration of pilocarpine is maintained in the bloodstream for at least 20 minutes such that composition provides relief from the pain and dry mouth caused by xerostomia, mucositis, or stomatotitis.

### FIELD OF THE INVENTION

The present invention relates to therapeutic agent delivery compositions, and more specifically to pilocarpine delivery compositions, and particularly to a pilocarpine chewing gum delivery composition that provides for improved pilocarpine absorption in the buccal cavity

### BACKGROUND OF THE INVENTION

Therapeutic agents or drugs are most frequently administered extravascularly. The majority are intended to act systemically, locally, peripherally on the central nervous system. Absorption is a pre-requisite to therapeutic activity. Delays or losses of drug during absorption contributes to variability in drug response and occasionally may result in sub-optimal effect, if not failure, of drug therapy.

The sequence of events for an oral composition includes absorption through the various mucosal surfaces, distribution via the blood stream to various tissues, biotransformation in the liver and other tissues, action at the target site, and elimination of drug or metabolites in urine or bile.

As a site for drug delivery, the oral cavity offers many advantages over other routes of drug administration. Oral mucosal drug delivery systems can be localized easily and are well accepted by patients, Rathbone, M., Drummond, B., and Tucker, I., Oral cavity as a site for systemic drug delivery, Adv. Drug Del. Rev., 13:1-22, 1994. Therefore, it is evident that the oral cavity can serve as a site for systemic drug delivery. The total surface area of the oral cavity is about 100 cm², Hoogstraate, A. J., Verhoef, J. C., Tuk, B., Pijpers, A., van Leengoed, L. A. M. G., Vheijden, J. H. M., Junjinger, H. E., and Bodde, H. E. Buccal delivery of fluorescein isothiocyanate-dextran 4400 and the peptide drug buserelin with glycodeoxycholate as an absorption enhancer in pigs, J. Control. Rel., 41:77-84, 1996. It has been shown that the buccal cavity offers excellent opportunities for systemic delivery of drugs. In general, drug delivery through this route has the advantages of preventing drugs from degradation in the gastrointestinal tract, avoiding first-pass effect, and bypassing gastrointestinal absorption. These advantages are the rationale for the present invention, which relates to absorption, and more particularly to absorption in the buccal cavity of the therapeutic agent pilocarpine.

The buccal mucosa offers several advantages for drug delivery for extended periods of time. The mucosa is well supplied with both vascular and lymphatic drainage. First-pass metabolism in the liver and pre-systemic elimination in the gastrointestinal tract are avoided. Chewing gum (bubble gum included) has been proven as a delivery vehicle for pharmaceutical and nutraceutical agents in recent years. From enormously successful smoking-cessation Nicorette® gums (Pharmacia) to the pain relief Aspergum® (Woodford) and from mineral-containing calcium gum to herb-containing Ginseng gum (Gumtech.com), it has increasingly become possible to deliver pharmaceutical agents with chewing gum. Additionally, transbuccal delivery compositions can successfully utilize other forms of carriers such as lozenges, quick dissolving tablets, chewing tablets, candy, gel, oral solutions, or other equivalent means. The combination of ingredients (compounds) can also be appropriate for delivery through such carriers.

The prior art basically deals with altering the environment of the mucosa to allow drug permeation. Examples of compounds used as oral mucosal permeation enhancers of various therapeutic agents are described in publications bellow.

23-lauryl ether—Oh, C. K. and Ritschel, W. A., Biopharmaceutic aspects of buccal absorption of insulin, Meth. Find Exp. Clin. Pharmacol., 12:205-212, 1990.

Aprotinin—Aungst, B. J. and Rogers, N. J., Site dependence of absorption-promoting actions of Laureth-9, Na salicylate, Na₂EDTA, and Aprotinin on rectal, nasal, and buccal insulin delivery, Pharm. Res., 5:305-308, 1988.

Azone—Kurosaki, Y., Hisaichi, S., Nakayama, T., and Kimura, T., Enhancing effect of 1-dodecycloheptan-2-one (Azone) on the absorption of salicyclic acid from keratinized oral mucosa and the duration of enhancement in vivo, Int. J. Pharm., 51:47-54, 1989.

Benzalkonium chloride—Siegel, I. A. and Gordon, H. P., Effects of surfactants on the permeability of canine oral mucosa in vitro, Tox. Lett., 26:153-157, 1985.

Cetylpyridinium chloride—Siegel, I. A. and Gordon, H. P., Surfactant-induced increase of permeability of rat oral mucosa to non-electolytes in vivo, Arch. Oral Biol., 30:43-47, 1985.

Cetyltrimethylammonium bromide—Kurosaki, Y., Hisaichi, S., Nakayama, T., and Kimura, T., Enhancing effect of 1-dodecylazacycloheptan-2-one (Azone) on the absorption of salicyclic acid from keratinized oral mucosa and the duration of enhancement in vivo, Int. J. Pharm., 51:47-54, 1989.

Cyclodextrin—Steward, A., Bayley, D. L., and Howes, C., The effect of enhancers on the buccal absorption of hybrid (BDBB) alpha-interferom, Int. J. Pharm., 104:145-149, 1994.

Dextran sulfate—Oh, C. K. and Ritschel, W. A., Biopharmaceutic aspects of buccal absorption of insulin, Meth. Find Exp. Clin. Pharmacol., 12:205-212, 1990.

Lauric acid—Coutel-Egros, A., Maitani, Y., Veillard, M., Machida, Y., and Nagai T., Combined effects of pH, cosolvent and penetration enhancers on the in vitro buccal absorption of propranol through excised hamster cheekpouch, Int. J. Pharm., 84:117-128, 1992.

Lauric acid/Propylene glycol—Aungst, B. J. and Rogers, N. J., Comparison of the effects of various transmucosal absorption promoters on buccal insulin delivery, Int. J. Pharm., 53:227-235, 1989.

Lysophosphatidylcholine—Zhang, J,, Niu, S., Ebert, C., and Stanley, T. H., An in vivo dog model for studying recovery kinetics of the buccal mucosa permeation barrier after exposure to permeation enhancers: apparent evidence of effective enhancement without tissue damage, Int. J. Pharm., 101:15-22, 1994.

Menthol—Coutel-Egros, A., Maitani, Y., Veillard, M., Machida, Y., and Nagai, T., Combined effects of pH, cosolvent and penetration enhancers on the in vitro buccal absorption of propranol through excised hamster cheekpouch, Int. J. Pharm., 84:117-128, 1992.

Methoxysalicylate—Oh, C. K. and Ritschel, W. A., Biopharmaceutic aspects of buccal absorption of insulin, Meth. Find Exp. Clin. Pharmacol., 12:205-212, 1990.

Methyloleate—Manganaro, A. M. and Wertz, P. W., The effects of permeabilizers on the in vitro penetration of propranolol through porcine buccal epithelium, Mil. Med., 161:669-672, 1996.

Oleic acid—Manganaro, A. M. and Wertz, P. W., The effects of permeabilizers on the in vitro penetration of propranol through porcine buccal epithelium, Mil. Med., 161:669-672, 1996.

Phosphatidylcholine—Coutel-Egros, A., Maitani, Y., Veillard, M., Machida, Y., and Nagai, T., Combined effects of pH, cosolvent and penetration enhancers on the in vitro buccal absorption of propranol through excised hamster cheek pouch, Int. J. Pharm., 84:117-128, 1992.

Polyoxyethylene—Oh, C. K. and Ritschel, W. A., Biopharmaceutic aspects of buccal absorption of insulin, Meth. Find Exp. Clin. Pharmacol., 12:205-212, 1990.

Polysorbate 80—Kurosaki, Y., Hisaichi, S., Hamada, C., Nakayama, T., and Kimura, T., Effects of surfactants on the absorption of salicylic acid from hamster cheek pouch as a model of keratinized oral mucosa, Int. J. Pharm., 47:13-19, 1988.

Sodium EDTA—Aungst, B. J. and Rogers, N. J., Site dependence of absorption-promoting actions of Laureth-9, Na salicylate, Na₂EDTA, and Aprotinin on rectal, nasal, and buccal insulin delivery, Pharm. Res., 5:305-308, 1988.

Sodium glycocholate—Aungst, B. J., Rogers, N. J., and Shefter, E., Comparison of nasal, rectal, buccal, sublingual and intramuscular insulin efficacy and the effects of a bile salt absorption promoter, The J. Pharmacol. Exp. Ther., 244:23-27, 1988.

Sodium glycodeoxycholate—Nakane, S., Kakumoto, M., Yulimatsu, K., and Chien, Y. W., Oramucosal delivery of LHRH: Pharmacokinetic studies of controlled and enhanced transmucosal permeation, Pharm. Dev. Tech., 1:251-259, 1996.

Sodium lauryl sulfate—Gandhi, R. and Robinson, J., Mechanisms of penetration enhancement for transbuccal delivery of salicylic acid, Int. J. Pharm., 85:129-140, 1992.

Sodium salicylate—Aungst, B. J. and Rogers, N. J., Site dependence of absorption-promoting actions of Laureth-9, Na salicylate, Na₂EDTA, and Aprotinin on rectal, nasal, and buccal insulin delivery, Pharm. Res., 5:305-308, 1988.

Sodium taurocholate—Wolany, G. J. M., Munzer, J., Rummelt, A., and Merkle, H. P., Buccal absorption of Sandostatin (octreotide) in conscious beagle dogs, Proceed. Intern. Symp. Control. Rel. Bioact. Mater., 17:224-225, 1990.

Sodium taurodeoxycholate—. Zhang, J., Niu, S., Ebert, C., and Stanley, T. H., An in vivo dog model for studying recovery kinetics of the buccal mucosa permeation barrier after exposure to permeation enhancers: apparent evidence of effective enhancement without tissue damage, Int. J. Pharm., 101:15-22, 1994.

Sulfoxides—Aungst, B. J. and Rogers, N. J., Comparison of the effects of various transmucosal absorption promoters on buccal insulin delivery, Int. J. Pharm., 53:227-235, 1989.

Various alkyl glycosides—Aungst, B. J., Site-dependence and structure-effect relationships for alkylglycosides as transmucosal absorption promoters for insulin, Int. J. Pharm., 105:219-225, 1994.

Permeation enhancers are used because the buccal cavity is a poor absorptive site of the alimentary tract. The buccal cavity lacks the typical villus-type of absorptive membrane of the intestine. Further, unlike the intestine, the junction between epithelial cells are tight. For a substance to be absorbed through the mucosal membrane of the buccal cavity, it has to be presented in a lipophilic form.

The mucosal membranes of the buccal cavity can be divided into five regions: the floor of the mouth (sublingual), the buccal mucosa (cheeks), the gums (gingiva), the palatal mucosa, and the lining of the lips. These oral mucosal regions are different from each other in terms of anatomy, permeability to drug, and their ability to retain a system for a desired length of time.

The ability of molecules to permeate through the oral mucosa is related to molecular size, lipid solubility, ionization and many other factors. Small molecules, less than about 100 daltons, appear to cross the mucosa rapidly. As molecular size increases permeability decreases rapidly. Lipid-soluble compounds are more permeable through the mucosa than are non-lipid-soluble molecules. In this regard, the relative permeabilities of molecules seem to be related to their partition coefficients. The degree of ionization of molecules, which is dependant on the pKa of the molecule and the membrane surface, also greatly affects permeability of the molecules. Maximum absorption occurs when molecules are un-ionized or neutral in electrical charge and absorption decreases as the degree of ionization increases. Therefore, charged drugs, such as ionized polypeptide based drugs, present a significant challenge to absorption through the oral musoca.

Most drugs are weak acids or weak bases and exist in solution as an equilibrium between the un-ionized and ionized forms. Increased accumulation of drug on the side of a membrane where pH favors greater ionization has lead to the pH partition hypothesis. According to this hypothesis, only un-ionized nonpolar drugs penetrate the membrane, and at equilibrium the concentrations of the un-ionized species are equal on both sides. The un-ionized form is assumed to be sufficiently lipophilic to traverse membranes. The fraction ionized is controlled by both the pH and the pKa of the drug according to the Handerson-Hasselbach equation.

Thus for acids,

pH=pKa+Log₁₀(Ionized concentration/Un-ionized concentration)

and for bases

pH=pKa+Log₁₀(Un-ionized concentration/Ionized concentration)

When pH is same as the pKa, equimolar concentrations of the un-ionized drug and ionized drug exist at that pH. At one pH unit lower than the pKa, the un-ionized to ionized ratio of an acid moiety is 91:9 and conversely, at one pH unit higher than the pKa, the un-ionized to ionized ratio of a base is 91:9. Further, at two pH unit lower than the pKa, the un-ionized to ionized ratio of an acid moiety is about 100:1 and conversely, at two pH units higher than the pKa, the un-ionized to ionized ratio of a base is 100:1. Stated differently, when the pH is two units lower than the pKa of an acid drug, almost all of the acid drug exists in a lipophilic form and when the pH is two units higher than the pKa, all the basic drug exists in ready to be absorbed lipophilic form.

The present invention makes use of functional buffer agents to increase absorption of therapeutic agents without altering the physiological environment of the mucosal lining of the oral cavity, by converting salts of basic or acidic drugs to non-ionized lipophilic forms. The oral delivery systems provided for in the present invention will allow the effective dosage to be substantially lowered as the actives are delivered more efficiently to the target site.

U.S. Pat. No. 6,344,222 describes a nicotine chewing gum delivery system in which a buffer system was used to increase the rate of release and absorption of nicotine. This patent uses only single buffer agents to facilitate a bi-phasic release of nicotine from the gum base and absorption across the buccal cavity membrane. In contrast, the present invention relies on a functional buffer system where two or more buffer agents are used in combination to promote a gradual and sustained change in pH in the buccal cavity. This gradual and sustained elevated pH improves the conversion of the salts of pilocarpine to its non-ionized lipophilic form thereby increasing the absorption of the pilocarpine across the membrane of the buccal cavity. In addition, the functional buffer system herein described avoids the abrupt change in pH experienced in a single buffer system. The buffer system of the present invention also contributes to an improved palatability of the therapeutic agent delivery composition.

U.S. Pat. No. 5,571,528 refers to a pilocarpine containing chewing gum for stimulating salivation. However, it does not disclose presence of a buffer system to convert the ionized pilocarpine salt to non-ionized lipophilic form to aid absorption.

U.S. Pat. No. 5,686,094 refers to controlled release formulation for the treatment of xerostomia using a polymeric delivery system which is formed by polycarbophil type composition with an active agent. The delivery system was able to achieve pharmacologically relevant plasma concentration by increasing the adherence time of the system to the mucosal membrane of the mouth.

U.S. Pat. No. 4,438,100 to Balslev et al. refers to a viscous artificial saliva containing a mucine and an oxidizing bactericide.

U.S. Pat. No. 4,209,505 to Mikhail refers to mouthwash for dry mouth relief, containing pilocarpine or a pilocarpine derivative. It is also noted therein that various types of diets have also been used (albeit unsuccessfully) in an attempt to alleviate xerostomia.

U.S. Pat. No. 4,151,270 to Ream et al. refers to a chewing gum composition formulated to stimulate salivation. The gum contains fructose and an organic acid such as adipic, ascorbic, citric, fumaric, lactic, malic or tartaric acids.

U.S. Pat. No. 4,938,963 refers to a method for treating xerostomia, comprising orally administering, to an affected individual, an amount of an eriodictyon fluid composition effective to alleviate the symptoms of dry mouth, the eriodictyon fluid composition comprising eriodictyon fluid extract and sweetener.

U.S. Pat. No. 4,917,674 refers to a medical device for the treatment of an individual suffering from xerostomia comprising two mouth moisturizing pads, each of which hold at least one sponge section wherein the sponge section is saturable with water for gradual dispensing of said water in the mouth.

U.S. Pat. No. 4,906,455 refers to a method for treating xerostomia wherein the patient chews, for a period of at least about 20 minutes, a gum containing a food-grade organic acid selected from the group consisting of adipic, fumaric, succinic, suberic, sebacic, azelic and pimelic acids.

U.S. Pat. No. 4,820,506 refers to a composition for promoting the production of human saliva consisting essentially of an aqueous liquid solution of water having dissolved therein:

(a) from about 2 to about 3 weight percent food-grade organic acidulent;

(b) a food-grade sweetener benign to stomic microflora selected from the group consisting of a sugar, a synthetic sweetener, and a reduced, sugar-related compound, and

(c) a saturated calcium phosphate solution.

The formulations of the present invention include, but are not limited to the following carriers: as lozenges, quick dissolving tablets, chewing tablets, candy, gel, oral solutions, chewing gums, or other equivalent means. The therapeutic agents used separately or in combination are pilocarpine acetate, and pilocarpine tartarate, pilocarpine hydrogen tartarate, pilocarpine bitartrate, pilocarpine hydrochloride, pilocarpine nitrate, pilocarpine dihydrochloride, pilocarpine sulfate, pilocarpine citrate, pilocarpine zinc chloride monohydrate, and pilocarpine salicylate, and its embodiment in other salt forms either in micronized or coarse form, and their embodiment in various salt forms. The

The pharmaceutical preparations as set forth in this invention, when formulated with the above listed therapeutic agents, the therapeutic agents used singularly or in combination with any of the other agents in the group would provide relief from orofacial complications, pain, and dry mouth caused by xerostomia, mucositis, or stomatotitis.

To reduce the dosage and therefore reduce side effects, the present invention provides, by use of functional buffers, a formulation that will neutralize the charge in situ in the oral cavity, restore the free form lipophilicity of pilocarpine and thereby facilitating in vivo absorption via increased permeability. The present invention will increase oral absorption allowing for the improved therapeutic effect at lower doses. Lower doses are generally preferable because undesirable side effects are decreased.

### OBJECTS AND ADVANTAGES

(a) to provide pharmaceutical compositions containing a functional buffer system for facilitating absorption of therapeutic agents in the buccal cavity.

(b) to provide a transbuccal delivery composition utilizing a carrier. Said carrier including either: chewing gum, lozenge, quickly-disintegrating tablet, chewable tablet, candy, gel or aqueous solution.

(c) to provide a transbuccal delivery composition for direct, immediate and controlled release of pharmaceutical compositions.

(d) to provide, by use of functional buffers, a formulation that will neutralize the charge in situ in the oral cavity, restore the free form lipophilicity of the active and thereby facilitating in vivo absorption via increased permeability.

(e) to provide functional buffer compositions capable of changing pH of the mouth to convert the salt form of the basic drug to a more absorbable free lipophilic form.

(f) to provide a transbuccal delivery system that will increase oral absorption allowing for the improved therapeutic effect at lower doses and thereby decreasing undesirable side effects.

### SUMMARY OF THE INVENTION

The present invention will allow for a more effective absorption of therapeutic agents across the oral cavity membrane through the use of a functional buffer system. Functional buffers will neutralize the charge in situ in the oral cavity, restore the free form lipophilicity of the active and thereby facilitate in vivo absorption via increased permeability. The functional buffers of the present invention are capable of changing the pH of the mouth for a sustained period of time in order to convert the salt form of pilocarpine to a more absorbable free lipophilic form; thereby providing for a transbuccal delivery composition for the direct, immediate and controlled release of the pilocarpine.

The claimed therapeutic agent delivery composition preferably comprises a pilocarpine constituent as the therapeutic agent, a carrier agent, and a functional buffer system whereby two or more buffer agents are combined with pilocarpine and administered orally as a composition with the carrier agent. The pilocarpine constituent can be either pilocarpine by itself, disbursed in a polymeric complex or any of the pharmaceutically acceptable salts of pilocarpine and or mixtures thereof. In preferred embodiments, the salts of pilocarpine include pilocarpine acetate, and pilocarpine tartarate, pilocarpine hydrogen tartarate, pilocarpine bitartrate, pilocarpine hydrochloride, pilocarpine nitrate, pilocarpine dihydrochloride, pilocarpine sulfate, pilocarpine citrate, pilocarpine zinc chloride monohydrate, and pilocarpine salicylate.

The functional buffer system, as part of the present invention, provides for raising of the pH in the buccal cavity for a sustained period of time. Raising the pH increases absorption of pilocarpine and effectively raises plasma concentrations of the active. The preferred embodiment of the invention for pilocarpine delivery system is to yield a pH in excess of at least about 7.5 inside the mouth, and even more desirably in the range from 8.0 to 10. A pH level of at least about 8.5 is particularly preferred inside the mouth.

As stated, the presence of the functional buffer system facilitates absorption of pilocarpine and in a preferred embodiment of the invention, the functional buffer system comprises a combination of two or more members selected from the group consisting of sodium carbonate, sodium bicarbonate, potassium carbonate, and potassium bicarbonate. Suitable pairs include sodium carbonate and sodium bicarbonate, potassium carbonate and potassium bicarbonate, sodium bicarbonate and potassium carbonate, and sodium carbonate and potassium bicarbonate. The members are combined in such a way that there is relatively more of the stronger base member than the weaker base member. When considered as a ratio, in particular embodiments, the ratio of the strong to weak member is about 10:1, even more preferred is the ratio of about 5:1, and a ratio of about 2:1 is particularly preferred.

In a preferred embodiment of the present invention the carrier agent is a chewing gum composition comprising a gum base matrix including at least one water soluble and one water insoluble portion, a pilocarpine constituent, and a functional buffer system. As stated above, the chewing gum composition raises the pH to the aforementioned desired levels in the buccal cavity within about 5 minutes after the onset of chewing. In preferred embodiments, the chewing gum composition contains a per dose serving of about 0.1 to 10 milligrams of a pilocarpine constituent, even more preferred about 1 to 10 milligrams, with about 1 to 5 milligrams being particularly preferred. In further embodiments, the chewing gum composition provides for a loaded pilocarpine concentration level in the blood stream of at least 10 to 100 nanograms of pilocarpine per milliliter of plasma.

In the preferred chewing gum composition of the present invention, the water insoluble portion of the gum matrix includes natural and synthetic polymers and rubbers and the water soluble portion consists of polyvinylacetate as the hydrophilic polymer. In another embodiment of the present invention, the hydrophobic polymer is polyisobutylene and the gum matrix comprises less than about 50% of the chewing gum composition. In further embodiments, at least one hydrophobic polymer may be selected from the group consisting of butadiene-styrene copolymers, butyl rubber, polyethylene, polyisobutyline and polyvinylesters. The pilocarpine chewing gum composition heretofore described can be formulated into any desired shape or size. The composition will take the shape of sticks or tabs, or any other form which is typically utilized by chewing gum manufacturers. The various formulations herein described are prepared using methods known in the confectionery industry for preparing commercial chewing gums. For example, the gum base is first softened by elevating its temperature, and adding softeners thereto by mixing. Next, any solid material (such as sweeteners in solid form) is combined therein by mixing. Finally, the active pilocarpine and any optional liquid material is also added by mixing. The composition is allowed to set and is shaped into serving sizes, which may be within the range of about 0.1 to 5.0 grams, for best results between 0.5 to 1 grams. In addition, each serving can be coated with an edible confectionery-type shell which may or may not contain any active pilocarpine or propofol ingredient. In yet another embodiment, the therapeutic agent delivery composition can also be presented using a lozenge, quick dissolving tablet, chewable tablet, candy, gel or aqueous solution as the carrier agent.

### BRIEF DESCRIPTION OF THE DRAWINGS

FIG. 1 is a graph of salivary pH achieved over time by separate chewing of exemplary embodiments of the present invention containing pilocarpine.

FIG. 2 is a graph of plasma pilocarpine concentration achieved over time in response to chewing of exemplary embodiment containing the functional buffer system, similar gum without the functional buffer system and the commercially available oral tablet of pilocarpine.

### DETAILED DESCRIPTION OF THE PREFERRED EMBODIMENTS

An example of the present invention is a delivery system containing pilocarpine and a buffer system to permit conversion of ionized pilocarpine salt to non-ionized lipophilic free base pilocarpine. While other forms may be contemplated by those skilled in the art and are within the scope set forth herein, the delivery system is best in the form of a chewing gum.

The chewing gum comprises a gum base matrix as a major component. The gum base matrix will include at least one gum base material which can be selected from the many water- and saliva-insoluble gum base materials known in the art. Illustrative examples of suitable polymers for gum bases include both natural and synthetic elastomers and rubbers, as well as mixtures thereof. Naturally-derived polymers include, for example, substances of plant origin like chicle, jelutong, gutta percha and crown gum. Synthetic elastomers such as butadiene-styrene copolymers, isobutylene and isoprene copolymers (e.g., “butyl rubber” in the art), polyethylene, polyisobutylene, polyvinylesters such as polyvinylacetate, and mixtures of any of the foregoing may be particularly useful.

In another preferred embodiment of the invention, the type of gum base utilized includes at least some butyl rubber (copolymer of isoprene and isobutylene), with additional amounts of polyisobutylene, and with polyvinylacetate (preferably PVA having a MW of approximately 12,000) also being present.

The gum base matrix (in whatever embodiment) will typically comprise from about 20 to 90% of the total chewing gum composition of the invention (unless otherwise stated, all percentages provided herein are weight percentages, based on either the total weight of the gum base matrix or of the final chewing gum composition, where noted). It is more preferred to utilize less than about 70% by weight of chewing gum base matrix material. In certain embodiments too much gum base may interfere with the release of the active ingredient, and additionally, may contribute to tackiness and poor mouth-feel of the final product. In an especially preferred embodiment of the invention, the chewing gum composition will contain about 50 to 60% of gum base matrix, and desirably about 55%. Of the foregoing amounts, about 25-75% thereof, more preferably about 30-60% thereof, will be the gum base polymer material(s) heretofore described.

The gum base matrix can additionally contain other ingredients well known in the art and selected from the group consisting of plasticizers and softeners to help reduce the viscosity of the gum base to a desirable consistency and to improve the overall texture and bite. These compounds are also noted for their emulsifying properties. As non-limiting examples, compounds such as lecithin, mono- and diglycerides, lanolin, stearic acid, sodium stearate, potassium stearate, glycerol triacetate, glycerol monostearate and glycerin are provided. Stearic acid, lecithin and mono- and diglycerides are particularly preferred. Plasticizers and softeners are desirable as part of the formulation because in addition to softening the primary gum base polymeric compound, they also seem to facilitate release of the active upon mastication. When added, the plasticizers and softeners will comprise from about 0.1 to 20% of the gum base matrix formulation, and more desirably will be within the range of about 5-15% thereof.

Waxes such as beeswax and microcrystalline wax, and fats/oils such as soybean and cottonseed oils are also contemplated as part of the gum base formulation. These compounds also function as softening agents. Typically, these compounds (either alone or in combination) will comprise from zero up to about 25% of the gum base matrix, and even more desirably will constitute less than about 20% of the gum base matrix, and more preferably will make up about 15-20% by weight of the gum base matrix. An especially desirable formulation will include a combination of microcrystalline wax and partially hydrogenated soybean oil in an approximate 1:2 weight ratio. A more exhaustive listing of these compounds, along with recommended weight percentages, can be found in any available industry reference.

Other materials which can be included as part of the gum base matrix include elastomer solvents. These are typically selected from the group consisting of rosin and resin material typically utilized in the confectionery chewing gum industry. Examples include methyl, glycerol, and pentaerythritol esters of rosins or modified rosins, such as hydrogenated, dimerized or polymerized rosins or mixtures thereof. More specific examples include pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin, such as polymers of alpha-pinene or beta-pinene, and terpene resins including polyterpene and mixtures thereof. Elastomer solvents can comprise from about zero to 75% of the gum base. It is preferable, however, to minimize or even eliminate the quantity of rosin/resin in the gum base. It is especially desirable not to exceed about 10% by weight of the gum base matrix with rosin/resin compound(s).

Filler material can also be present in the gum base matrix as part of the composition of the invention. This material is further selected to enhance the chewability of the final chewing gum composition. In at least some embodiments, certain filler material can also enhance the release and absorption of pilocarpine and other similar basic drugs such as lidocaine, benzocaine, bupivacaine, etidocaine, mepivacaine, pramoxine, prilocaine, procaine, proparacaine, ropivacaine, tetracaine, chloroprocaine, and their embodiment in various salt forms. Those fillers which are substantially non-reactive with other components of the final formulation are also preferred. Desirable filler materials will therefore include calcium carbonate, magnesium silicate (talc), as well as dicalcium phosphate, and any mixtures thereof. Particularly preferred can be dicalcium phosphate. Other metallic mineral salts can also be utilized as filler material, as for example alumina, aluminum hydroxide, and aluminum silicates, provided they possess the characteristics heretofore set forth. Filler material will typically comprise about 0.1 to 30% of the gum base matrix, and more preferably will be within the range of about 10 to 20% thereof.

Trace amounts of standard industry preservatives such as butylated hydroxy toluene (BHT) can also be present in amounts less than about 0.1% or so of the gum base.

Further provided, as part of the pilocarpine chewing gum formulation of the invention, is at least one bulk sweetener. This material is added to the composition to impart improved palatability to the chewing gum composition, and thereby provide a pleasant chewing experience to help in masking any unpleasant tastes presented by therapeutic ingredients. The “sweetener” may or may not be perceptibly sweet. Examples of sweeteners include those compounds selected from the group consisting of saccharide material such as the mono-, di-, tri- and polysaccharide materials available in the industry, including oligomers, and oligosaccharides. As non-limiting examples, sugars such as sucrose, glucose (corn syrup), dextrose, invert sugar, fructose, and mixtures thereof can be useful. Less or non-sweet sugars and polysaccharide material such as maltodextrin and polydextrose can also be utilized. In certain embodiments of the invention, however, “sugar-free” or “non-sucrose” formulations can be especially desirable. Thus, natural and synthetic non-saccharide-based sweeteners can be selected from the group consisting of saccharin and its various salts such as the sodium and calcium salts, cyclamic acid and its various salts, dipeptide sweeteners, chlorinated sugar derivatives such as sucralose, dihydrochalcone, and sugar alcohols such as sorbitol, sorbitol syrup, mannitol, xylitol, hexa-resorcinol and the like, including mixtures of any of the foregoing, are contemplated for use herein. Hydrogenated starch hydrolysate, (lycasin), and the potassium, calcium and sodium salts of 3,6-dihydro-6-methyl-1-1,2,3-oxathiazin-4-on3-2,2-dioxide are also within the scope of the invention as sweetener material. Of the foregoing, sorbitol and xylitol are particularly preferred, either alone or more desirably in combination. Xylitol can be desirable because of its non-cariogenic or anti-cariogenic properties.

The bulk sweetener(s) will make up about 5 to 75% of the chewing gum composition of the invention. It is more preferable to include one or more sweeteners within the range of about 25 to 40% of the final formulation, even more desirably about 30 to 35% of the gum composition.

In addition to the bulk sweetening material, the composition of the invention also comprises one or more flavoring agents. These can be selected from any of the industry-available natural and synthetically-derived food and pharmaceutical flavors in whatever form. Especially preferred are those materials which impart a cooling and/or vaporizing sensation to the consumer upon mastication of the gum. As non-limiting examples, peppermint, spearmint, wintergreen, cinnamon, menthol and menthone flavors, oils and derivatives are desirable. Food and pharmaceutical grade coloring agents available throughout the industry can also be utilized. Any of the foregoing flavor and coloring agents, either alone or in combination will typically comprise from about 0 to 10% of the chewing gum composition, more preferably from about 0.1 to 5%, and even more desirably about 2 to 3% thereof. It is also within the scope of the invention that the formulations specifically not contain any adjunct flavors or colors.

The delivery system of the invention also comprises one or more active ingredients. “Pilocarpine” as that term is used herein refers to pilocarpine salts and can include, for example, pilocarpine acetate, and pilocarpine tartarate, pilocarpine hydrogen tartarate, pilocarpine bitartrate, pilocarpine hydrochloride, pilocarpine nitrate, pilocarpine dihydrochloride, pilocarpine sulfate, pilocarpine citrate, pilocarpine zinc chloride monohydrate, and pilocarpine salicylate. Of the foregoing, pilocarpine hydrogen tartarate and pilocarpine bitartrate can be especially suitable. In addition, “pilocarpine” also includes the solid complex of either therapeutic agent bound to an ion exchange or other polymer disbursing systems. Particularly, a cation ion exchanger for pilocarpine.

In still another embodiment of the invention, an efficacious release of pilocarpine is obtained if a salt thereof is utilized in conjunction with a butyl rubber-based gum base matrix (together with PVA), as heretofore described. Thus, pilocarpine may be particularly preferred in conjunction with this butyl rubber-based gum material. The inclusion of some PVA in the butyl rubber-based formulation can further act synergistically on absorption of pilocarpine.

In still another embodiment of the invention, an efficacious release of pilocarpine is obtained if a salt thereof is utilized in conjunction with a Pharmagum S gum base matrix (together with or without PVA), as heretofore described. Pharmagum is a mixture of a polyol(s) and/or sugars with a chewing gum base. This gum is manufactured under cGMP conditions and complies with the Food Chemicals Codex specifications as well as with the FDA, so they can be considered as “Generally Recognized As Safe” (GRAS). The literature on Pharmagum and its chemistry is readily available from its manufacturer, SPI Pharma Group, 321 Cherry Lane, New Castle, Del. 19720-2780. Thus, pilocarpine may be particularly preferred in conjunction with this butyl rubber-based gum material. The inclusion of some PVA in the butyl rubber-based formulation can further act synergistically on absorption of pilocarpine.

Per dose serving, hereinafter described, of the pilocarpine chewing gum composition of the invention will, for best results, contain about 0.1 to 10 milligrams of pilocarpine (as measured in its free base form). More desirably, the amount of pilocarpine will be within the range of about 1 to 10 milligrams, and even more preferably be within the range of about 1 to 5 milligrams. In some embodiments, it may be particularly preferred to include about 1-4 milligrams of pilocarpine in a serving, with perhaps 2.5 milligrams being especially desirable. Of the foregoing amounts, the skilled artisan may choose to add extra pilocarpine, for best results, up to about 10-25% or so by weight. This extra amount may be regarded as overage, that is, the amount which may be expected to be “washed away” or otherwise not released or absorbed during mastication. As a weight percentage, the total amount of pilocarpine (in whatever chosen form, measured as per its free base form) will typically comprise about 0.01 to 10%, and more preferably be within the range of about 0.1 to 1% of the chewing gum composition. It may be especially desirable to utilize about 0.20 to 0.8% of pilocarpine by weight, with about 0.25% being especially preferred. The foregoing percentages will vary depending upon the particular source of pilocarpine utilized, the amount of pilocarpine the skilled artisan desires to include in the final formulation, as well as on the particular release rate of the pilocarpine or pilocarpine resin complex desired.

Another ingredient included as part of the pilocarpine chewing gum delivery system of the invention is a buffer composition or system. Buffering agents are those compounds that assist in release and conversion of the pilocarpine salts (ionized pilocarpine) to pilocarpine free base (unionized pilocarpine). Passage of actives across the mucous membranes inside the mouth to the bloodstream and to target tissues is due primarily to passive diffusion of the unionized form of the active. To be effective the buffer material should be released in sufficient amounts with the release of the active to create a basic or alkaline pH environment inside the mouth, thereby facilitating effective delivery to target organs. Consequently, conversion of salts of pilocarpine in the chewing gum into free base pilocarpine in mouth saliva is an important step in providing sufferers of dry mouth with adequate blood levels of pilocarpine. Buffer compounds assist with this conversion by raising the pH and thereby facilitating pilocarpine absorption.

The functional buffer systems comprise a combination of two or more of the following salts: sodium carbonate, sodium bicarbonate, potassium carbonate, potassium bicarbonate, potassium citrate and mono basic potassium phosphate, or mixtures thereof, are particularly preferred if the therapeutic agent is pilocarpine. In certain embodiments, especially with butyl rubber-based gum base formulations, a combination of sodium carbonate and sodium bicarbonate or potassium carbonate and potassium bicarbonate or sodium bicarbonate and potassium carbonate or sodium carbonate and potassium bicarbonate may be most desirable as the functional buffer composition. The buffering agent will comprise about 0.1 to 10% of the pilocarpine chewing gum formulation, and desirably will be within the range of about 0.5 to 5% thereof. In particular, about 1 to 3% quantity of buffer may be especially desirable in the final formulation. On a weight basis, the buffer will usually comprise about 1.0-100 milligrams in a 1 gram serving of final product. More preferably, there will be about 10-50 milligrams, and typically about 30 milligrams. The optimal buffering system will consist of a combination of at least two buffer salts. Typically the proportion of the relatively more basic buffer salt of the two buffers should be more than that of the less basic buffer salt. For example, when the functional buffering system consist of sodium carbonate and sodium bicarbonate, proportionally sodium carbonate should be more than sodium bicarbonate. Desirably, the ratio of the relatively more basic buffer salt and the less basic buffer salt should be 10 to 1, in particular, the desired ratio should be 5 to 1 and typically it is 2 to 1. The buffer raises the pH of the milieu inside the buccal cavity.

The preferred embodiment of the invention for the pilocarpine delivery composition is to yield a pH in excess of at least about 7.5 inside the mouth, and even more desirably in the range from 8.0 to 10. A pH level of at least about 9.0 is particularly preferred inside the mouth. As stated, the presence of the buffering composition facilitates absorption of pilocarpine. Thus, the buffer system as part of the present invention provides highly effective plasma concentrations of pilocarpine.

Typically, chewing gum formulations comprise three major components. These are a water-insoluble portion, a water-soluble portion, and a flavoring agent. The water-insoluble portion is called gum base. The water-soluble portion is mainly a combination of sweeteners. When a gum is chewed, the sweeteners and flavoring agents are gradually extracted out with saliva, leaving the gum base as a cud in the end. Thus, the chewing process is actually a controlled-release process of sweeteners and flavors. A well formulated gum provides a smooth, pleasant texture and a long, steady release of sweeteners and flavors. Structurally, gum base is a complex mixture of ingredients, including food-grade elastomer, plasticizers, texture agents, waxes, lipids, and emulsifiers. FDA allows the use of only a handful of elastomers such as butyl rubber, natural rubber (including certain natural gums), polyisobutylene (PIB), polyvinylacetate (PVAc), and styrene-butadiene rubber (SBR) as chewing gum elastomer. The elastomer component in natural gums is polyisoprene (both cis-1,4 and trans-1,4). The cis isomer is amorphous and therefore more rubbery at ambient temperatures, while the trans isomer is semicrystalline at ambient temperatures, with a melting range of 50-70° C. The inclusion of the buffer system in the gum formulation results in a chewing gum that delivers more of the therapeutic agent than the corresponding solid oral dose tablet intended for gastrointestinal dissolution and release of the active and/or the corresponding chewing gum formulation without the functional buffer systems.

The various embodiments of the pilocarpine delivery system—chewing gum composition heretofore described may be formulated into any desired shape or size. Preferably, the composition will take the shape of sticks or tabs, or any other form which is typically utilized by chewing gum manufacturers. The various formulations herein described are prepared using methods known in the confectionery industry for preparing commercial chewing gums. For example, the gum base is first softened by elevating its temperature, and adding softeners thereto by mixing. Next, any solid material (such as sweeteners in solid form) is combined therein by mixing. Finally, the active pilocarpine or propofol and any optional liquid material is also added by mixing. The composition is allowed to set and is shaped into serving sizes, which may be within the range of about 0.1 to 5.0 grams, preferably about 0.5 to 2 grams. In addition, each serving may be coated with an edible confectionery-type shell which may or may not contain any active pilocarpine or propofol ingredient. The pharmaceutical preparation can also be presented as a lozenge, quick dissolving tablet, chewable tablet, candy, gel or aqueous solution.

In another embodiment of the invention, there is provided a chewing gum delivery system in which a gum base matrix material, in the form of granulates, has one or more of the active pilocarpine substances interspersed among the granulates. The gum base granulates together with the active(s) are compressed together to yield the final formulation. The gum base matrix may be material as heretofore described, i.e. that which facilitates release of the active (as for example that having a hydrophilic moiety, or a butyl rubber-based moiety), or may be other gum matrix material known in the art. For example, a low moisture, non-aqueous gum base matrix having a high degree of hydrophobicity may be utilized in certain formulations. In certain situations, the gum base matrix material and the pilocarpine can have different, somewhat incompatible moieties so that the pilocarpine is not strongly retained by the gum base matrix, and can be released more easily.

In this embodiment of the invention, wherein gum base granulates are used, it is especially desirable that the pilocarpine be thoroughly dispersed among the gum base granulate matrix, but preferably not be contained within the granulates themselves. It may also be desirable that the pilocarpine substantially enrobe or surround each of the individual granulates as well.

To therefore prepare this embodiment of the pilocarpine chewing gum composition of the invention, the procedures set forth in U.S. Pat. No. 4,405,647 may be especially helpful to the skilled artisan. Briefly stated, the gum base material may be melted or softened using one or more of the softening agents, plasticizers and/or solvent and filler materials heretofore described. The sweeteners and flavors, whether processed via flash-flow processing or other traditional mixing methods, are then admixed into the gum base. This is accomplished by comminuting the gum base material together with the water-soluble ingredients in a bed or blender within a gaseous medium at room temperature, as described in the aforementioned U.S. Pat. No. 4,405,647. This material is continuously pulverized and thereby chopped into much smaller particles. To prevent adherence of the resultant particles to one another, additional filler or bulking material may be added like lubricants, glidants and other tableting and compression aids well known in the pharmaceutical industry, such as for example, silica gel or calcium carbonate. Granules of any desired size and shape may be obtained upon the introduction of a standard mess screen to separate the particulates once formed.

The next step in forming the final chewing gum composition involves adding the pilocarpine active to the formed particulates. This is done by admixing the pilocarpine, whether in free form or encapsulated as heretofore described, with the pulverized materials so as to substantially disperse the pilocarpine among the particulates. In a preferred mode, the pilocarpine may be added along with the tableting, lubrication or other compression aids. The active material thus becomes substantially entrapped in the multitude of spaces between the individual gum particles. Upon thorough mixing by any suitable device, the materials are then compressed and compacted in a tablet press or other suitable device. In this way the pilocarpine is sandwiched in the voids in between the compressed particulate gum granulate material. The active substance is thoroughly dispersed between and throughout the resulting matrix. The active is thus “external” to the gum base material itself. The result is an external delivery system for pilocarpine. In a particularly preferred embodiment, the active material(s) together with the non-actives, heretofore described, are provided in a substantially non-liquid format. That is, the formulation of the invention according to this embodiment is preferably substantially 0% liquid.

Other possible physical embodiments of the pilocarpine chewing gum composition of the invention include, for example, various centerfill configurations. In these embodiments the gum base matrix will at least partially surround a centerfill. The centerfill will contain one or more of the active pilocarpine substances. The centerfill may be a liquid or semi-liquid material and preferably will be low fat or fat free. In addition to the active(s), the centerfill may contain one or more sweeteners and/or flavorants as heretofore described. A combination of saccharide material, flavoring, polyol and edible gel material is one example of a centerfill. One or more of the active ingredient(s) and/or the sweeteners and flavorants, etc. may be encapsulated as previously set forth, and then incorporated into the centerfill.

The centerfill embodiments may be prepared using methods known in the confectionery and chewing gum industries. For example, U.S. Pat. No. 3,806,290 describes a method for forming centerfill chewing gum by extruding a hollow-centered rope of chewing gum through an orifice having a pair of concentric conduits extending there through. A centerfill material is fed through the inner conduit to the hollow center upstream through a space between the inner and outer conduits. The centerfill rope of chewing gum is passed to a sizing unit having a plurality of pairs of rollers for progressively decreasing a cross-sectional dimension of the gum rope. The plurality of pairs of rollers includes at least one vertical pair of rollers having vertically aligned axes of rotation and overlapping lower flange portions. Ramp means are provided for guiding the gum rope above the roller flange portions upon entry of the gum rope between the vertical pair of rollers. Other methods of forming centerfill chewing gum known in the art may also be utilized.

The centerfill embodiment may be particularly desirable wherein immediate release of the pilocarpine active is particularly desired. Encapsulating the active ingredient(s) in this embodiment may help to taste-mask those actives which provide an undesirable organoleptic sensation. Other than the centerfill portion, it is preferred that the formulation ingredients of this embodiment also be substantially liquid-free, or about 0% liquid.

A further embodiment will include a gum base matrix containing pilocarpine, together with a centerfill containing pilocarpine and the buffer compositions as well. The pilocarpine and buffer compositions in the centerfill can be released quickly to relieve an individual's dry mouth, while the matrix can release pilocarpine and the buffer compositions over time thereafter to maintain pilocarpine levels in the blood.

The pilocarpine chewing gum containing the corresponding functional buffer systems with or without the aforementioned local anesthetics can be used for treatment of dry mouth, caused by a variety of clinical and pathological reasons. The dry mouth can be as a result of Sjogren's syndrome, xerostomia, stomatitis and mucositis. Also, a common manifestation of chemotherapy or radiation in cancer patients is dry mouth. Dry mouth can also manifest itself as a side effect of anti-depressant, anti-hypertension, anti-histamine, anti-retroviral, anti-tussive, nasal decongestant therapy. After introduction of a serving size piece of the gum composition into the mouth, the consumer will chew the gum as is normally done with any non-medicated type of chewing gum for about 20-30 minutes, but at approximately an average rate of about 10-45 chews per minute. The gum is then discarded. Care should be exercised, however, to avoid overdosing. A serving of the pilocarpine chewing gum delivery system of the invention is designed to cause a loaded pilocarpine concentration level in the bloodstream of at least about 10 to 70 nanograms of pilocarpine per milliliter of plasma.

A serving of the pilocarpine chewing gum delivery system of the invention is designed to cause a loaded pilocarpine concentration level in the bloodstream of at least about 10 to 100 nanograms of pilocarpine per milliliter of plasma.

Each individual possesses unique factors effecting bioavailability of a drug (pharmaceutical composition) following oral dosing. The delivery systems described herein allows for a higher plasma levels and therefore offers a much better therapeutic approach for the management of a wide range of ailments.

For a drug to be absorbed, it has to permeate across the mucosal membrane. Three major sources of variation in permeability of a given membrane to a drug are molecular size, lipophilicity and charge. Of the three variables, lipophilicity and charge are most relevant to this invention. Lipophilicity is often characterized by partition between oil and water. Small lipid-soluble unionized drugs tend to penetrate lipid membrane with ease. Charge is the other major constraint to transmembrane passage. There is considerable variation in the impedence of different membranes to charged molecules, but the effect of the charge is, with few exceptions is always negative.

The present invention will, by use of functional buffers in the formulation, neutralize the charge in situ in the oral cavity, restore the free form lipophilicity of the active and there by facilitating in vivo absorption via increased permeability. The functional buffers are capable of changing the pH of the mouth and thereby convert the salt form of the basic drug to a more absorbable free lipophilic form.

The following examples illustrate various preferred embodiments of the invention, but are not to be construed as limiting the scope thereof:

### EXAMPLES

Examples of the gum form were prepared and tested for effectiveness and performance vis-à-vis pH of the buccal mileau and relative bioavailability of pilocarpine from the embodiment of this invention and the commercially available tablet (Salagen®). Two different gum bases served as ingredients for the examples. The first exemplary embodiment of the present invention consisted of gum base commercially available Dreyco® Base. This gum base is sugar free and non-acidic in nature. It consists of elastomers, plasticizers, resins, emulsifiers and waxes. The second exemplary embodiment of the present invention was made using a direct compressible gum base commercially available as Pharmagum™ S. Pharmagum™ S is a mixture of polyol(s) with a gum base.

The pilocarpine in all of these examples can be provided after mixing with a disbursing agent, usually, but not limited to, copolymers such as poly(vinyl alcohol), poly(2-hydroxyethyl methacrylate), poly(ethylene oxide) and polyvinylpyrrolidone and polymers of cellulose such as hydroxypropylmethyl cellulose, hydroxypropyl cellulose, hydroxyethyl starch and sodium carboxymethyl cellulose.

In the following examples, the exemplary pilocarpine chewing gum formulation was compared to certain control formulations as well as the commercial formulation available as Salagen® tablets. Comparisons were made in the ability of the exemplary chewing gum to change the pH of the buccal milieu and relative bioavailability of pilocarpine from the exemplary embodiment of this invention and a similar gum without the buffer system. The pH of saliva was measured after collecting saliva resulting from chewing of the exemplary embodiment by human subjects. For this purpose, a serving size of gum (approximately 1.0 gram each) was chewed at a timed rate of approximately 1 chew per second for 3 seconds on one side of the cheek and transferred to the other cheek of the mouth at the fourth second. The chewing session lasted for a total of 5, 10 and 15 minutes. The saliva resulting from the chewing was collected every 30 seconds. Each serving of gum contained approximately 5 mg. of pilocarpine. The plasma concentrations of pilocarpine were measured from blood samples collected from a three way cross-over study in healthy male smokers. The smokers were selected due to their exposure and familiarity with Nicorette® gum. Plasma pilocarpine concentrations were determined by validated gas chromatography/mass spectrometry. The reference standard was pilocarpine hydrochloride, USP, purity 100%. The internal standard was ethylpilocarpine hydrochloride, purity 99.7%. The internal standard was stored frozen a approximately −20° C. In this method, pilocarpine hydrochloride and its internal standard were extracted into methylene chloride:hexane (1:1) from human plasma made basic by the addition of a sodium tetraborate buffer. The organic layer was transferred to a clean test tube and evaporated to dryness. The residue was derivatized using heptafluorobutyric acid anhydride and triethylamine in toluene at an elevated temperature. After multiple cleanup and concentration steps, the resulting extract was analyzed by gas chromatography on a J&W DB-17 column using mass spectrometric detection. The calibration cures for pilocarpine hydrochloride in human plasma were linear in the concentration range from 1.00 to 50.0 ng/ml with correlation coefficients of 0.9994 or greater using power regression analysis. The precision of the method was determined from the relative standard deviation (RSD) of concentration values for four quality-control sample pools. The RSD of the method range from 2.9% to 5.2% for pilocarpine hydrochloride at the four quality-control sample levels. The RSD of the slopes of the calibration cures were within 1.5%. The accuracy of the method in human plasma was determined by comparing the means of the measured concentration of the quality-control samples with their normal concentrations. The mean values were within 6.7% of their expected values for pilocarpine.

### Example 1

In this example, the salivary pH study was conducted in 3 healthy male volunteers using the exemplary formula according to one embodiment of the invention, and a similar gum formulation without the buffer system. The exemplary formula contained pilocarpine hydrochloride (approximately 5 mg of pilocarpine base). The Formula A chewing gum that is exemplary of the embodiment of this invention contained the functional buffer system consisting of a combination of sodium carbonate 1.5% (15 mg per 1 gram serving dose) and sodium bicarbonate 0.75% (7.5 mg per 1 gram serving dose). This formula also contained Pharmgum™ S as the gum base (90% or 900 mg per 1 gram serving dose). In addition, it also contained xylitol (5% or 50 mg per 1 gram serving dose) and talc/magnesium stearate (1:1), 2.25% or 22.5 mg per-serving dose. The control Formula B without the buffer system consisted of 92.25% of Pharmagum™ S (922.50 mg per 1 gram serving dose). This formula also contained pilocarpine hydrochloride (approximately 5 mg of pilocarpine base) xylitol (5% or 50 mg per 1 gram serving dose) and talc/magnesium stearate (1:1), 2.25% or 22.5 mg per serving dose. The chewing gum tablet was prepared by direct compression after mixing in a tableting machine. With the exception of talc/magnesium stearate, the other ingredients are weighed and mixed for agglomeration in a high speed ribbon blender. The mix is then dried in a fluid bed dryer and lubricated with talc/magnesium steerage powder. The lubricated mixed agglomerate is subsequently compressed at a speed of 10 mm/minute in a press fitted with 15 mm flat face F tooling.

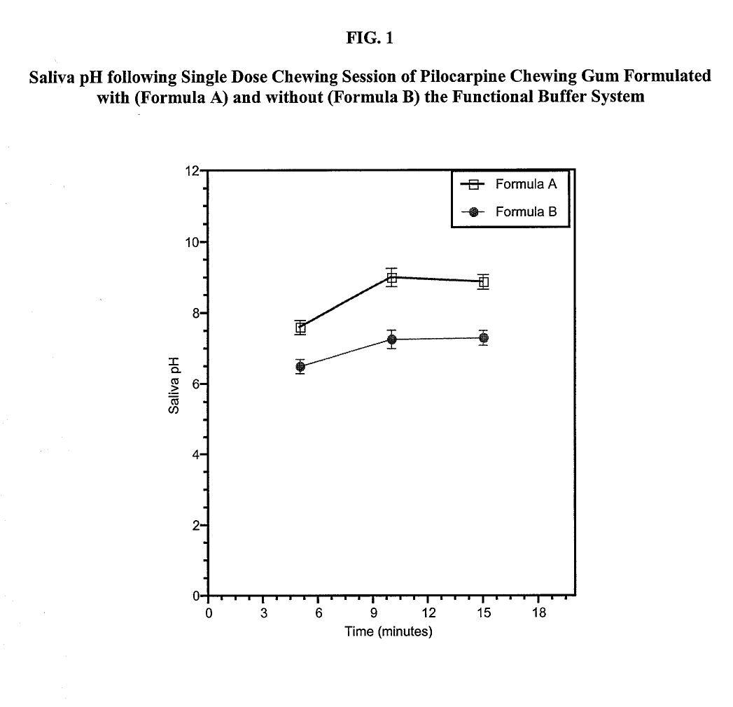

The results are set forth in FIG. 1. As can be seen, the gum containing the buffer system was able to distinctly change the pH of the saliva. Formula A, consisting of pilocarpine and the sodium carbonate/sodium bicarbonate buffer system is progressively able to increase the pH from the initial neutral pH. This demonstrates that a buffering system as part of a buccal delivery system is a facilitator of an optimum pH environment inside the mouth. Such a buffering system can be adjusted to deliver a desirable amount of buffer. This, in turn, facilitates the absorption of a pH dependent compound such as pilocarpine.

### Example 2

In this example, the relative bioavailability of pilocarpine in 6 healthy male volunteers was compared following single dose administration of the exemplary gum containing pilocarpine and the functional buffer system, the control gum also containing pilocarpine but formulated without the buffer system and the commercially available Salagen tablet. The Formula E chewing gum that is exemplary of the embodiment of this invention contained the functional buffer system consisting of a combination of sodium carbonate 1.5% (15 mg per 1 gram serving dose) and sodium bicarbonate 0.75% (7.5 mg per 1 gram serving dose). This formula also contained Dreyco™ as the gum base (71% or 710 mg per 1 gram serving dose) and pilocarpine (0.5% or 50 mg equivalent of the base per serving dose). In addition, it also contained xylitol (25.5% or 255 mg per 1 gram serving dose) magnesium oxide 0.75% or 7.5 mg per serving dose. The control Formula F without the buffer system consisted of 73.25% of DreycoTm (732.50 mg per 1 gram serving dose). This formula also contained pilocarpine hydrochloride (approximately 5 mg of pilocarpine base) contained xylitol (25.5% or 255 mg per 1 gram serving dose) magnesium oxide 0.75% or 7.5 mg per serving dose.

The chewing gum formulations were prepared by melting the gum base at 85° C. in a pre-heated insulated vessel. All other ingredients are added and mixed in to the melt using a rotary blender. The melt is subsequently kneaded to a semi solid mass, which is subsequently extruded as 1″ inch long chewing gum sticks.

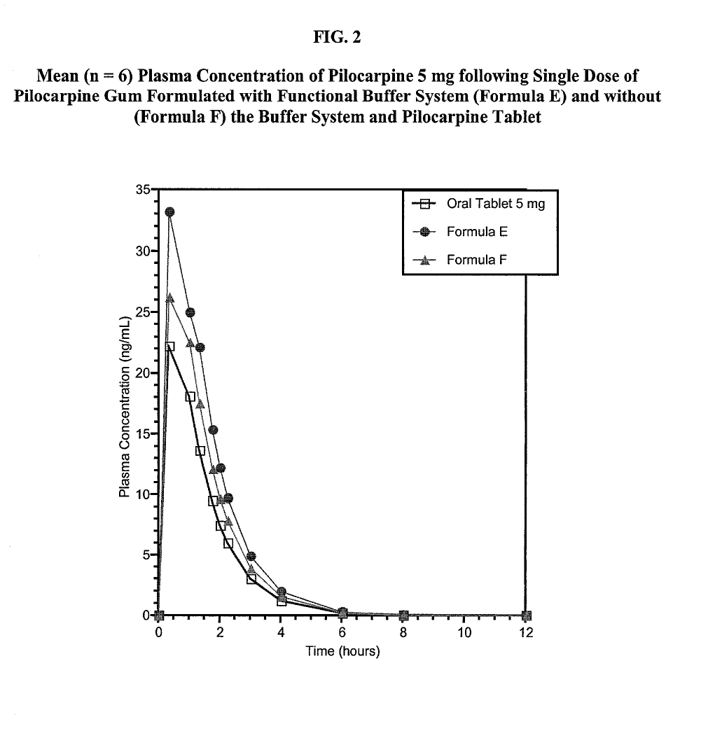

The results are set forth in FIG. 2. As can be seen, the plasma data clearly demonstrates that the bioavailability of pilocarpine from the gum formulation containing the buffer system is about 26% and 52% more than that of the gum formulation without the buffer system and the commercially available tablet, respectively.

### Example 3

In this example, a quick dissolving tablet was prepared as exemplary of one embodiment of the invention. The exemplary quick dissolving Formula G tablet contained the functional buffer system consisting of a combination of sodium carbonate 1.5% (7.5 mg per 0.5 gram serving dose) and sodium bicarbonate 0.75% (3.25 mg per 0.5 gram serving dose). The formula also contained quick dissolving ingredient commercially available as Pharmaburst™ 80% or 400 mg per 0.5 gram serving dose, pilocarpine 0.5% or 2.25 mg per 500 mg serving dose, aspartame 0.15% or 0.75 mg per 0.5 gram serving dose, xylitol 17% or 85 mg per 0.5 gram serving dose and menthol flavor 1% or 5 mg per 0.5 gram serving dose. The tablets were mixed and manufactured to yield tablets with average hardness of about 20N and disintegration time of 20 seconds.

Formula G is an embodiment of an example where the functional buffer system can be used for enhancing the absorption of the primary therapeutic agent, pilocarpine.

### Example 4

The exemplary formula, Formula I, contained pilocarpine hydrochloride (0.5% or approximately 5 mg as free base per 1 gram serving dose) in combination with benzocaine (1.0% or approximately 10 mg as free base per 1 gram serving dose). The Formula I chewing gum that is exemplary of the embodiment of this invention contained the functional buffer system consisting of a combination of sodium carbonate 1.5% (15 mg per 1 gram serving dose) and sodium bicarbonate 0.75% (7.5 mg per 1 gram serving dose). This formula also contained Pharmgum™ S as the gum base (89% or 890 mg per 1 gram serving dose). In addition, it also contained xylitol (5% or 50 mg per 1 gram serving dose) and talc/magnesium stearate (1:1), 2.25% or 22.5 mg per serving dose. The chewing gum tablet was prepared by direct compression after mixing in a tableting machine. With the exception of talc/magnesium stearate, the other ingredients are weighed and mixed for agglomeration in a high speed ribbon blender. The mix is then dried in a fluid bed dryer and lubricated with talc/magnesium steerage powder. The lubricated mixed agglomerate is subsequently compressed at a speed of 10 mm/minute in a press fitted with 15 mm flat face F tooling.

Formula I is an embodiment of where the principle therapeutic agent, pilocarpine, can be combined with local anesthetic in a chewing gum formulation.

The foregoing exemplary embodiments provide a convenient, reliable, practical, and relatively painless system for delivering an active. Notably, the delivery system of the present invention is capable of rapidly achieving a pharmacologically effective concentration of the active (e.g., pilocarpine) in the bloodstream (e.g., within 30 minutes or more desirably within 10 minutes, or in some cases, within 1-2 minutes), and is also capable of keeping the concentration of the active in the bloodstream at or near the pharmacologically effective concentration for at least 20 minutes after chewing of the delivery system begins, or more desirably about 30 minutes to about 50 minutes after chewing begins.

Similarly, the exemplary dosage amount of about 5 milligrams for pilocarpine is not a limitation of the present invention. It will be appreciated from the foregoing teachings that alternative dosage amounts can be provided (e.g., 0.1-10 milligrams of pilocarpine, or more desirably, 1-4 milligrams of pilocarpine) by suitably modifying the composition that defines the delivery system.

Although the invention has been described in reference to its preferred embodiments those of ordinary skill in the art may make modifications therein without departing from the scope and spirit of the invention which is claimed below. It is expected that certain changes or modifications to the invention disclosed herein may be effected by those skilled in the art without departing from the true spirit and scope thereof as set forth in the claims and the accompanying specification.

What is claimed is:

1. A therapeutic agent delivery composition for systemic, oral administration of a pilocarpine constituent, said composition comprising:
a) a pilocarpine constituent;
b) a carrier agent; and
c) a functional buffer system, whereby two or more buffer agents are combined with said pilocarpine constituent and administered orally as a composition with said carrier agent.

2. The composition of claim 1, wherein said composition provides for maximum plasma concentration of pilocarpine in about one hour or less after oral administration.

3. The composition of claim 1, wherein said functional buffer system raises the pH in the mouth to about 7.5 or more within 5 minutes after administration.

4. The composition of claim 1, wherein said pilocarpine is in the form of at least one member selected from the group consisting of pilocarpine by itself, pilocarpine disbursed in a polymeric complex, and the pharmaceutically acceptable salts of pilocarpine.

5. The composition of claim 4, wherein said pilocarpine comprises at least one member selected from the group consisting of pilocarpine acetate, and pilocarpine tartarate, pilocarpine hydrogen tartarate, pilocarpine bitartrate, pilocarpine hydrochloride, pilocarpine nitrate, pilocarpine dihydrochloride, pilocarpine sulfate, pilocarpine citrate, pilocarpine zinc chloride monohydrate, and pilocarpine salicylate.

6. The composition of claim 1, where in said carrier agent is a gum base matrix including at least one substantially hydrophobic polymer and at least one hydrophilic polymer.

7. The composition of claim 6, wherein said gum base matrix comprises polyvinylacetate as said hydrophilic polymer and at least one hydrophobic polymer member selected from the group consisting of water-insoluble, natural and synthetic elastomers, polymers and rubbers.

8. The composition of claim 7, wherein said gum base matrix comprises butyl rubber and polyisobutylene, as said hydrophobic polymer, said gum base matrix comprising less than about 50% of said composition.

9. The composition of claim 8, wherein said hydrophobic polymer member comprises at least one member selected from the group consisting of butadiene-styrene copolymers, butyl rubber, polyethylene, polyisobutylene and polyvinylesters.

10. The composition of claim 6, wherein said gum base matrix alternatively comprises a directly compressible mixture of water-insoluble, natural and synthetic elastomers, polymers and rubbers and polyols such as mannitol, sorbitol, xylitol, and/or monosaccharides such as glucose and mannose.

11. The composition of claim 10, wherein said polymers comprise about 25-75% of said gum base matrix.

12. The composition of claim 11, wherein said polymers comprise about 50-60% of said gum base matrix, and said gum base matrix comprises about 25-75% of said composition.

13. The composition of claim 12, further comprising at least one bulk sweetener selected from the group consisting of mono-, di-, tri- and polysaccharides, including oligomers and oligosaccharides, and natural and synthetic non-saccharide-based sweeteners, dipeptide, chlorinated sugar derivatives or sugar alcohols, hydrogenated starch hydrolysate, (lycasin), the potassium, calcium and sodium salts of 3,6-dihydro-6-methyl-1-1,2,3-oxathiazin-4-on3-2,2-dioxide.

14. The composition of claim 3, wherein said buffer system comprises two or more members selected from the group consisting of sodium carbonate, sodium bicarbonate, potassium carbonate and potassium bicarbonate.

15. The composition of claim 14, wherein said buffer system comprises a combination of members selected from the group consisting of sodium carbonate and sodium bicarbonate, potassium carbonate and potassium bicarbonate, sodium bicarbonate and potassium carbonate, and sodium carbonate and potassium bicarbonate.

16. The composition of claim 15, wherein the combination of members are combined in such a way that there is relatively more of the stronger base member than the weaker base member.

17. The composition of claim 16, wherein the combined members are combined in a ratio of about 10:1.

18. The composition of claim 16, wherein the combined members are combined in a ratio of about 5:1.

19. The composition of claim 16, wherein the combined members are combined in a ratio of about 2:1.

20. The composition of claim 15, further comprising at least one filler material as described herein.

21. The composition of claim 15, comprising at lest one local anesthetic as described herein.

22. The composition of claim 1, wherein said carrier agent is a lozenge dosage form.

23. The formulation in claim 1, wherein said carrier agent is selected from a group consisting of quick-dissolving tablet, chewable tablet, candy, gel and aqueous solution.

24. A chewing gum composition for systemic, oral administration of a pilocarpine constituent, said composition comprising:
a) a pilocarpine constituent;
b) a gum base matrix, said gum base matrix including at least one water insoluble portion and a water soluble portion; and
c) a functional buffer system, whereby two or more buffer agents are combined such that said pilocarpine constituent is administered by the chewing gum composition.

25. The composition of claim 24, wherein said composition raises the pH of the buccal cavity to about 7.5 or more within about 5 minutes after the onset of chewing and wherein said pilocarpine constituent comprises at least one member selected from the group consisting of pilocarpine by itself, pilocarpine disbursed in a polymeric complex, and the pharmaceutically acceptable salts of pilocarpine.

26. The composition of claim 25, wherein said composition raises the pH of the buccal cavity to about 8.0 to 10.0 within about 5 minutes after the onset of chewing.

27. The composition of claim 24, wherein the per dose serving of said pilocarpine in said chewing gum composition is about 0.1 to 10 milligrams.

28. The composition of claim 24, wherein the per dose serving of said pilocarpine in said chewing gum composition is about 1 to 10 milligrams.

29. The composition of claim 24, wherein the per dose serving of said pilocarpine in said chewing gum composition is about 1 to 5 milligrams.

30. The composition of claim 25, wherein a serving of said chewing gun composition provides for a loaded pilocarpine concentration level in the bloodstream of at least about 10 to 100 nanograms of pilocarpine per milliliter of plasma.

31. A chewing gum composition for systemic, oral administration of a pilocarpine constituent, said composition comprising:
a) a pilocarpine constituent, the per dose serving of said pilocarpine in said chewing gum composition is about 0.1 to 10 milligrams, and wherein the chewing gum composition provides for a loaded pilocarpine concentration level in the bloodstream of at least about 10 to 100 nanograms of pilocarpine per milliliter of plasma;
b) a gum base matrix, said gum base matrix including at least one water insoluble portion and a water soluble portion; and
c) a functional buffer system, whereby two or more buffer agents are combined such that said pilocarpine constituent is administered by the chewing gum composition, and wherein said composition raises the pH of the buccal cavity to about 7.5 or more within about 5 minutes after the onset of chewing.
